(19) United States
(12) Patent Application Publication
(10) Pub. No.: US 2003/0124604 A1
(43) Pub. Date: Jul. 3, 2003

(54) Method for amplification of molecular bio-assay signals

(76) Inventor: Fengchun Ye, Edmonton (CA)

Correspondence Address: HOGAN & HARTSON L.L.P., 500 S. GRAND AVENUE, SUITE 1900, LOS ANGELES, CA, 90071-2611, US

(21) Appl. No.: 10/299,066

(22) Filed: Nov. 18, 2002

(30) Foreign Application Priority Data
Nov. 23, 2001 (CA) 2,363,703

(51) Int. Cl. C12Q 1/68; C12P 19/34

(52) U.S. Cl. 435/6; 435/91.2

(57) ABSTRACT

Disclosed are reagents and a method for effective in vitro amplification of bio-assay signals. The method makes use of a pair of “end-to-end” complementary oligonucleotide primers to continuously form a double-stranded and highly repetitive hybrid molecule. Since this hybrid molecule is covalently linked and added to the probing molecule in the bioassays, and is also compatibly labeled, it can amplify the detection signals hundreds of times within a short period. The method can have very broad applications in bioassays using nucleic acid hybridization, immunochemical detection, and other specific interactions between two molecules or between molecules and cells.

### FIELD OF THE INVENTION

The present invention relates to a method for covalent linkage of a specially designed sequence to probing molecules used in bio-assays/detection reactions, and subsequent amplification of this sequence to fortify the initial signals from the target-bound probing molecules in the bio-assays. The disclosed method is a very useful technique that can have wide applications in molecular biology, chemistry, biotechnology, medical diagnosis, and forensic science.

### BACKGROUND OF THE INVENTION

A big challenge in molecular detection or other molecular interaction based bio-assays is low sensitivity of the detection system. Very often, it is necessary to amplify the detecting signals in order to detect target molecules of extremely low quantity.

A means of amplifying nucleic acid molecules is particularly of great value because a lot of molecular detection methods and bio-assays use molecular hybridization of nucleic acids (DNA-DNA, DNA-RNA, or RNA-RNA), including Dot blot hybridization, Southern and Northern blot hybridization, in situ hybridization, and DNA chip screening, etc. Applications of nucleic acid detection are extremely broad, ranging from fundamental gene mapping, genotyping, and gene expression analysis, to diagnosis of infectious diseases, cancer, and genetic diseases. In addition, nucleic acid molecules can be easily conjugated to other molecules such as proteins. Conjugation of nucleic acid to other molecules is essential for their own detection, and/or for having complementary and fortified signals when such molecules themselves are used as probes in detection.

Several useful methods have been developed to amplify specific nucleic acid molecules. The earliest DNA amplification method was the polymerase chain reaction (PCR), which involves a DNA polymerase, a 5′ end and a 3′ end primer to target specific DNA, and dNTP (dATP, dCTP, dGTP, and dTTP) to replicate the target DNA in an exponential manner (U.S. Pat. No. 4,965,188). For each cycle of amplification, the reaction begins with converting double-stranded target DNA into single-stranded DNA under denaturing conditions (high temperature). This is then followed by separate annealing of two specific oligo primers to the two complementary but already disassociated templates, and subsequent polymerization by the DNA polymerase to form two new double-stranded DNA molecules under lower temperatures. The discovery and usage of a thermo stable DNA polymerase, Taq DNA polymerase, has led to full automation of the PCR process, rendering this DNA amplification method extremely powerful. A similar method, known as ligase chain reaction (LCR), uses DNA ligase and primers that are target DNA-specific to amplify the target DNA exponentially (Landegren, et al., Science, 241: 1077-1080 (1988). Kalin, et al., Mutation Research, 283 (2): 119-123 (19920. Abravaya, et al., Nucleic Acids Res., 23 940: 675-682 (1995). Thermo stable ligase has also been found and used to perform automatic LCR amplification.

Several other DNA amplification methods that are performed under isothermal (37° C.) conditions have also been developed. These include self-sustained sequence replication (3SR), nucleic acid sequence based amplification (NASBA), strand displacement amplification (SDA), amplification with Q.beta replicase, and rolling circle amplification (RCA) methods (Fahy, et al., PCR Methods, 1:25-33 (1991). Guatelli, et al., Proc. Natl. Acad. Sci. USA, 87:1874-1878. Compton, Nature, 359: 91-92 (1991). Walker, et al., Nucleic Acids Res., 20: 1691-1696 (1992). Kievits, et al., J. Virol. Methods, 35: 273-286 (1991). Miele, et al., J. Mol. Biol., 171:281-295 (1983). Mills, J. Mol. Biol., 200: 489-500 (1988). Lizardi, et al., Nat Genet, 19225-232 (1998). also refer to U.S. Pat. No. 5,409,818, 5,455,166, 5,714,320, 5,854,033, 5,871,921, 6,077,668, 6,143,495, 6,261,773, 6,287,824, and 6,291,187). The 3SR and NASBA methods involve three enzymes: reverse transcriptase, RNase H, and a DNA-dependent RNA polymerase. The enzymes are used to amplify specific RNA and/or DNA target molecules exponentially. The SDA method is based on a restriction endonuclease nicking its recognition site and a polymerase extending the nick at its 3′ end, displacing the downstream strand. The displaced sense strand serves as a target for an antisense reaction and vice versa, resulting in exponential growth of the target molecule. The RCA method mimics the DNA replication mechanism of some viruses. A DNA polymerase, primed by a primer, reads off of a single promoter around a circle of DNA. This continuously rolls out linear stretches of the circle. At first it creates a copy of itself, then it continues to create a concatenated string of multiple copies. This linear RCA reaction (LRCA) can run for several days, producing millions of copies of the small circle sequence that are covalently attached. An improvement on the LRCA method is the use of exponential RCA (ERCA), with additional primers that anneal to the LRCA product strand. Thus, double-stranded DNA can be produced, and exponential amplification can occur via strand displacement reactions referred to as HRCA (Lizardi, et al. Nature Genetics, 9: 225-231 (1998).

All these DNA amplification methods are very efficient and powerful to amplify selected specific target DNA or RNA molecules. However, each has some shortcomings. For instance, one disadvantage of PCR, 3SR, NASBA and SDA is that the amplified products are dispersed in the reaction solution, not covalently attached to the target. In a case such as in situ hybridization detection, it is important that the amplified signals are attached to the target. If they are dispersed in the solution, they will be washed away. In other words, the amplified signals risk being lost during washing steps if they are not covalently linked to the target. Another disadvantage of these methods is that they can't efficiently amplify very large molecules. By comparison, RCA has some advantages over these methods, such as covalent linkage of all amplified signals to the target, and the ability to amplify very large molecules. Nevertheless, all these in vitro amplification procedures require functional enzymes to replicate the target molecules. These enzymes can be difficult and costly to produce, and the amplification reactions are fairly complicated. In addition, except RCA, all other methods are very difficult to be used for detection/bio-assay involving other types of molecules such as proteins.

### BRIEF SUMMARY OF THE INVENTION

Disclosed are reagents and a method for efficient in vitro amplification of signals in molecular detections such as nucleic acid hybridization, antibody/antigen immunoassay, or other specific molecule-molecule binding assays. The method uses three oligonucleotide primers. The first primer, referred to as reference primer, is 25-50 bases long in general, and is covalently linked to the probe molecules (DNA, RNA, antibody, etc.) used in the detection/bio-assays. The second primer, Amplifier I, is a symmetrical molecule, with its 5′ half sequence fully complementary to the reference primer and its 3′ half sequence fully complementary to the 3′ half of a third primer named Amplifier II. Like Amplifier I, Amplifier II is also symmetrical, with its 5′ half identical to the reference sequence and thus also fully complementary to the 5′ half of Amplifier I. Depending on how the probe molecules are labeled and which detection method is used, the two amplification primers are also compatibly labeled. This labeling can be done using either radioactive or non-radioactive methods such as fluorescence, biotin, and others.

The signal amplification procedure makes use of the ability of the two “end-to-end” complementary amplification primers to hybridize and form highly repetitive sequences. It follows the last wash step of the molecular detection assays. Amplifier I is first incubated and hybridized with the reference primer linked to the probe molecules that are specifically bound to the targets. Amplifier II is then added, which hybridizes with the 3′ half of amplifier I. The protruding single-stranded 5′ half of the added Amplifier II will continue to hybridize with the 5′ half of Amplifier I in the buffer, and the “walk” continues endlessly until the incubation is stopped. Although the amplification is not exponential, the “linear addition” process can effectively amplify the detection signals hundreds of times within a short period.

The method of the present invention (referred to herein as Non-Enzymatic Amplification (NEA)) is a simple but efficient procedure for signal amplification. It does not require any enzyme to amplify the involved nucleic acid molecules, and can be used for signal amplification of any bio-assay that is based on molecular interactions. These molecular interactions include those between DNA-DNA, DNA-RNA, RNA-RNA, DNA/RNA-protein, protein-protein, molecule-cells, and any other probe-target interactions. Thus, the method can be used to amplify signals of a variety of different probe molecules, including nucleic acids, peptides, proteins, and other chemicals. Moreover, the method can be used to simultaneously amplify signals of multiple probes/targets in a single detection/bio-assay. Another important distinction between all the previously described amplification methods and the method of the present invention is that the latter does not amplify the target molecule itself. Rather, it amplifies additive nucleic acid signals that are integrated and added to the target-specific probing molecules. Thus, the present invention has the advantages of being highly useful in much broader applications, involves an easier procedure, and has lower cost, no risk of contamination, and more flexibility, especially in molecular detection and diagnosis assays.

### BRIEF DESCRIPTION OF THE DRAWINGS

FIG. 1 depicts the designs of the reference primer, the Amplifier I primer, and the Amplifier II primer.

FIG. 2 illustrates steps of the amplification procedure.

FIG. 3 demonstrates how the method amplifies signals in detections using nucleic acid hybridization.

FIG. 4 demonstrates how the method amplifies signals in detections using antibody or other proteins as probes.

### DETAILED DESCRIPTION OF THE INVENTION

As used herein, a “target” in a molecular detection/bio-assay refers to the molecule or the organism in a testing sample that the assay tries to identify and/or quantify. It can be a molecule, such as nucleic acid, protein, peptide, carbohydrate, lipid, hormone, antibody, antigen, and a chemical; it can also be an organism, such as a virus, bacterium, or cell, etc.

A “probe” in a bio-assay refers to the molecule that the assay uses to specifically identify and/or qualify the target in a testing sample. The probe is usually a molecule, such as nucleic acid, protein, peptide, carbohydrate, lipid, hormone, derivatives thereof or analogs thereof, that has intrinsic capabilities to specifically bind to its target. The probe molecules are usually labeled with a fluorescent label, a phosphorescent label, an enzymatic label, a chemical label (such as biotin or digoxigenin), or a radioactive label, for specific signal detection. p A “oligonucleotide” primer refers to a sequence-defined and length-defined nucleic acid or analog thereof.

The disclosed method is a simple but very effective procedure to substantially amplify signals in molecular detection. Unlike other DNA amplification technologies, such as polymerase chain reaction (PCR), ligase chain reaction (LCR), and rolling cycle amplification (RCA), etc., the disclosed method does not use any enzyme such as DNA polymerase or DNA ligase.

The disclosed method requires three nucleic acid primers: a reference primer and two amplification primers (Amplifier I and Amplifier II). None of these primers should have significant homology with the probe/target molecules in nucleic acid hybridization assays. However, in other methods of molecular detection such as immunoassays, this requirement does not apply, and a universal set of primers can be used.

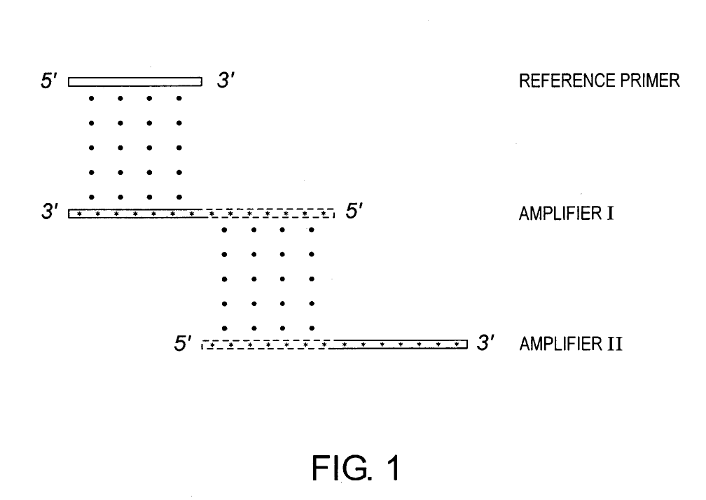

A. Designs of Primers (FIG. 1):

The first primer, the reference primer, generally 25-50 bases long (note: longer sequences might also be used, as far as they can be produced). This can be either a DNA or RNA sequence. This sequence should be relatively G+C rich, without any ability to form internal “hair-pin” structures. This sequence might be derived from the “multiple cloning sites” of a DNA cloning vector, in which an interested DNA fragment is inserted. More specifically, the reference primers can be chosen from the two regions near the two ends of the cloned DNA fragment insert. When the interested DNA fragment is cut out and used as a probe for molecular hybridization, the selected reference primers from the vector sequences should be included and attached to the probe DNA fragment. This can be realized either by PCR amplification of the chosen area using specific primers, or by cutting the fragment out of the vector with specific restriction enzymes. The same can be done for cRNA probe preparation when using the insert-carrying plasmid as a template for in vitro transcription with T3, T7 or SP6 RNA polymerase. If synthesized olignucleotides are used as probes in the detection/bio-assays, a reference sequence can be included and attached to each probe molecule during primer synthesis. When total RNAs or mRNAs are used to make cDNA probes, in the case of gene chip screening assays, for example, the reference primer sequence can be attached to the 5′ end of the polyT primer that is used in the reverse transcription reaction. As such, the resulting cDNA molecules (probe) will have the reference primer sequence attached at their 5′ end.

If proteins, such as antibodies, protein A, protein G, avidin, streptavidin, etc. are used as probes in the detection assays, the selected reference primer should be covalently immobilized to the probe molecules. A number of strategies can be used for covalent coupling of nucleic acid molecules to proteins. Ultraviolet cross-linking can effectively immobilize DNA molecules to proteins (Jang, et al., J. Immuno. 145: 3353-3359 (1990).). Chemical cross-linking also can be very efficient. One of these methods is coupling 5′ Thiol oligos to sulfo-GMBS activated and β-mercaptoethanol treated proteins (Schweitzer, et al., Proc. Natl. Acad. Sci. USA, 97:10113-10119 (2000).). Another method is coupling 5′ amine-modified oligos to proteins through an amine crosslinker reagent. Glutaraldehyde can also be used to couple 5′amine-modified oligos to proteins. In this case, the 5′ amine-reference primer is incubated with 2% glutaraldehyde. After ethanol precipitation or column purification is carried out to remove the un-used glutaraldehyde, the primer-glutaraldehyde complex is incubated with proteins, resulting in the following structure: reference primer-N═CH—(CH₂)₃—CH═N-protein. The oligonucleotide conjugated proteins are finally subjected to gel filtration purification to remove free oligonucleotides. Coupling DNA primers to other types of probe molecules is also possible. Chemical reactions depend on the nature of the probe molecules.

The other two primers in the disclosed method are amplification primers: Amplifier I and Amplifier II. Depending on the reference primer, these primers are generally 50-100 bases long. They can be either DNA or RNA sequences. Again, longer sequences can be used for the amplification process, as long as they can be produced. These sequences can be either synthetic primers, can be made through recombinant DNA technology, or can be the products of in vitro transcription or reverse transcription reactions. The primer Amplifier I is a symmetrical molecule, with its 5′ half sequence fully complementary to the reference primer, and its 3′ half sequence fully complementary to the 3′ half of the third primer, Amplifier II. When choosing the primer sequences, one must make sure that there isn't any possibility for the primers to form internal “hair-pin” structures. Like Amplifier I, Amplifier II is also symmetrical, and its 5′ half is identical to the reference sequence and fully complementary to the 5′ half of Amplifier I.

Depending on how the probe molecules are labeled and which detection method is used, the two amplification primers are compatibly labeled. For instance, if the probe is given a radioactive label such as ³²P, the two amplification primers should also be labeled with the same radioactive element. This will ensure that the amplified signals and the original signals from the probe molecules are entirely compatible and can be detected by the same method or device. This makes it possible for the two readings to be added together. Such labeling systems can be either radioactive methods or non-radioactive methods that use fluorescence, biotin, and other labeling chemicals. For radioactive labeling of the primers, either T4 polynucleotide kinase or terminal deoxynucleotidyl transferease can be used for end labeling. For non-radioactive labeling, fluorescence, biotin, digoxigenin, or other labeling molecule-conjugated nucleotides can be directly incorporated into the sequence during the primer synthesis.

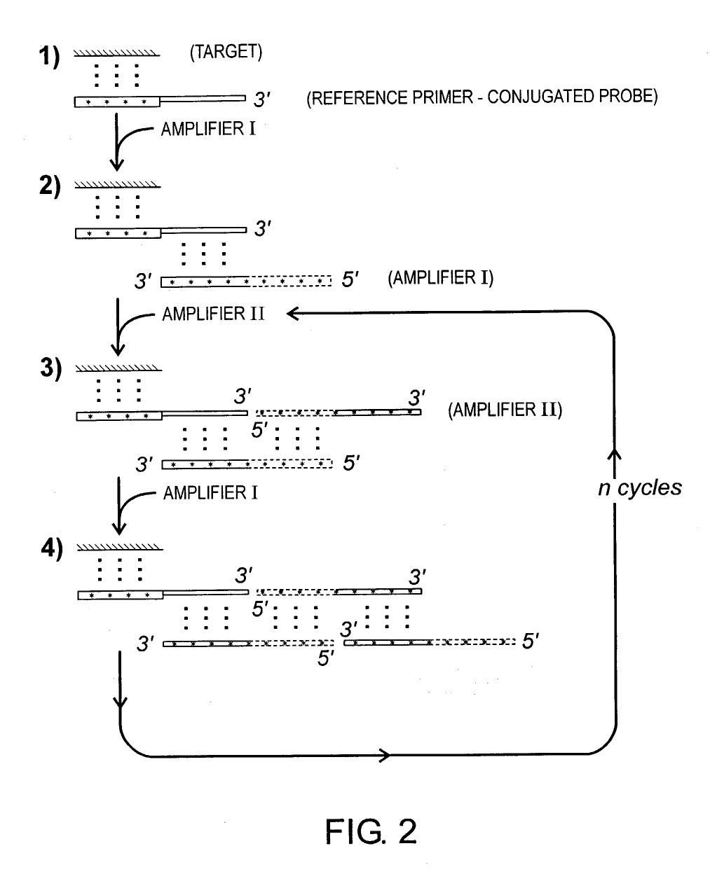

B. Amplification Procedure (FIG. 2):

The signal amplification procedure follows the last wash step of the molecular detection assays. At this point, only the probe molecules are specifically bound to the targets. To fortify detection signals through the disclosed method, Amplifier I is first incubated with the “probe-target” complex, hybridizing with the reference primer that has been linked to the probe molecules in the previous steps. This step can be done in a regular DNA hybridization buffer or in a particular buffer compatible with the bio-assays. The key is that all solutions must be nuclease-free. This step can take place within a few minutes. Amplifier II is then added, which hybridizes with the 3′ half of Amplifier I. The protruding single-stranded 5′ half of the added Amplifier II will continue to hybridize with the 5′ half of free Amplifier I in the hybridization solution. The outcome is the formation of a double-stranded hybrid molecule with another protruding single-stranded 3′ half of Amplifier I, which will continue to hybridize with free Amplifier II. As such, the “walk” continues endlessly until the incubation is stopped.

Ideally, the amplification process is done in a programmed manner, with the two amplification primers separately added to the amplifying molecule. This is because the two amplification primers are “end-to-end” complementary sequences. Co-incubation of these two primers may generate linear and circular hybrids of various sizes in the hybridization solution. Consequently, this may not be in favor of the continuous addition of the two primers to the extending hybrid (amplifying signal) attached to the “probe-target” complex. In other words, separate incubation of the “probe-target” complex with the two amplification primers would keep the two primers constantly at high concentration. This would facilitate the hybridization and extension of the hybrid bound to the “probe-target” complex. A disadvantage of such separate addition is that longer time is required to have a substantial amplification because each addition step takes at least one minute. However, if an appropriate concentration of primers is used, the whole amplification process may be done with the two primers mixed in a single tube and carried out as a single step. In this case, incubation time can be largely reduced. In conclusion, although the amplification is not an exponential but a linear addition process, it can effectively amplify the detection signals hundreds of times within a short period.

With regard to molecular hybridization conditions for the signal amplification steps, Tm of all the primers must be calculated for different salt concentrations. Stringency conditions (mainly temperature and salt concentration) for hybridization and washing can be accordingly estimated and used for the process. All steps in signal amplification are carried out under sufficiently stringent conditions. In addition, although the disclosed method is basically a non-enzymatic process, we have found that the reaction, when conducted in an appropriate buffer, can be significantly improved in the presence of a DNA ligase, such as T4 DNA ligase. Therefore, whenever possible, it is suggested to include a certain amount of DNA ligase in the solutions containing the two amplification primers, in order to achieve higher signal amplification.

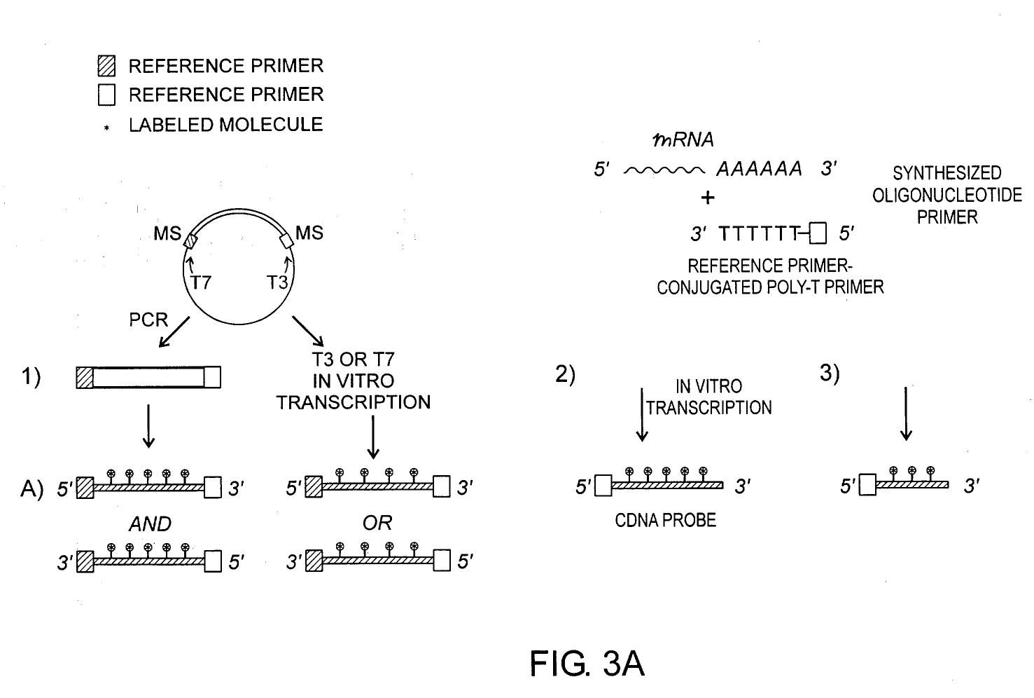

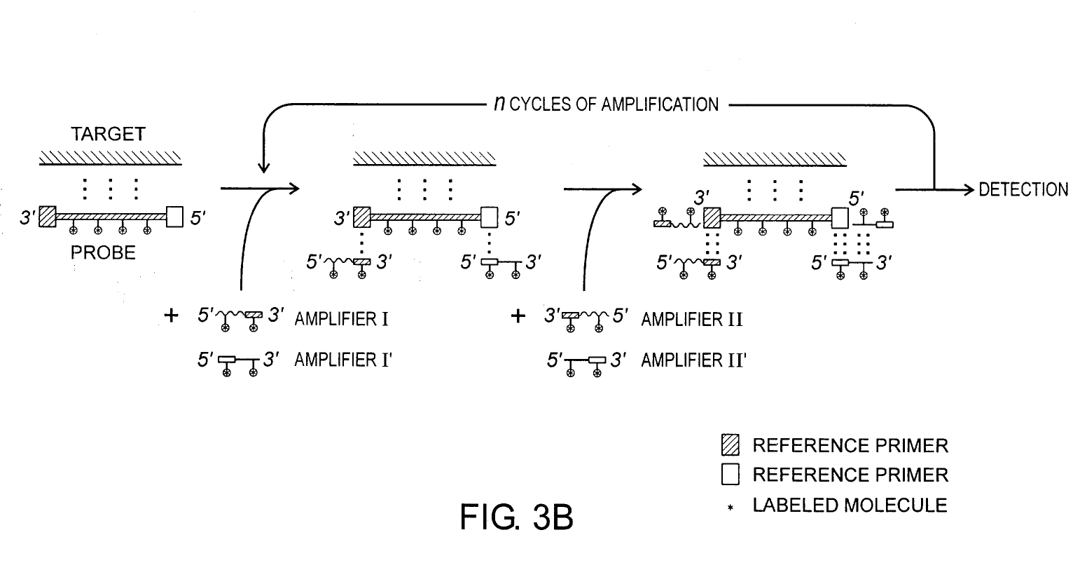

C. Application of the Disclosed Method to Nucleic Acid Hybridization Assays (FIG. 3):

This kind of application includes a number of molecular hybridization bio-assays, such as Southern blot, Northern blot, in situ hybridization on tissue, cells, or chromosomal spreads, and molecular hybridization on DNA chips, etc. The disclosed method can be particularly useful when the detection signals are very weak without signal amplification.

FIG. 1A illustrates how DNA, RNA, and oligonucleotide probes can be prepared to have the reference primer(s) attached to one or both ends. When the probe is made from a DNA fragment cloned in a plasmid or phage vector, it would be advantageous to have two reference primers, each attached to one end of the insert (probe DNA). This would allow simultaneous signal amplification at both ends of the probe DNA molecule. The two reference primers can be chosen from the vector sequences near the two ends of the probe DNA, in the multiple cloning sites (MS) regions. A radioactively or non-radioactively labeled DNA probe can be prepared in the presence of a labeled-dNTP (dCPT, dGTP, dATP, or dTTP) by PCR amplification of the chosen area. Otherwise, the chosen fragment (including the reference sequences at both ends of the insert) can also be cut out of the vector with appropriate restriction enzymes, and labeled with whatever appropriate methods. Alternatively, cRNA (complementary RNA) probes can also be made if promoter sequences for T3, T7, or SP6 RNA polymerases are available upstream of the multiple cloning sites from either direction. If mRNAs (messenger RNAs with polyA tails) are the templates for probes, for instance, when preparing cDNA probes to hybridize with DNA chips for gene expression analysis, a randomly formulated reference primer can be directly attached to a polyT sequence. It can be attached at its 5′end during oligo primer synthesis. This polyT primer is then used to prime first-strand cDNA synthesis, in the presence of a labeled-dNTP and reverse transcriptase. Finally, if synthesized oligo primers are used as probes, the reference primer can be directly attached to the probe primer during primer synthesis and labeling.

FIG. 1B illustrates steps of molecular hybridization and signal amplification after hybridization. Using the cloned DNA probe (FIGS. 1A-1) as an example, after hybridization to the target and washing away non-specific hybridization signals (noise or background signals), and prior to final detection, a further signal amplification step is carried out using the disclosed method. Since the two reference primers are different sequences, one way to do signal amplification at both ends of the probe molecule is to have two pairs of amplification primers, with one pair fitting one reference primer and the other pair fitting the other reference primer. The advantage of this strategy is that there will not be any interference between the amplifying signals at the two ends. The disadvantage is that it requires four amplification primers. To avoid such complication, another strategy involves the use of a single pair of amplification primers that are designed as follows:

Amplifier I: its 5′ half to be complementary to the reference primer at the 5′ end of the probe molecule, and its 3′ half to be complementary to the reference primer at the 3′ end of the probe molecule;

Amplifier II: its 5′ half to be identical to the reference primer at the 5′ end of the probe, and its 3′ half to be identical to the reference primer at the 3′ end of the probe.

The advantage of this strategy is that it requires only one pair of amplification primers. The disadvantage is that at some point the extended hybrids from the two ends of the probe may form a closed circle, preventing further extension of the amplifying signals. This occurs because the two amplification primers are “end-to-end” complementary. From this point of view, the “four-primer” strategy is a better choice.

In practice, the two different Amplifier I primers can be mixed and prepared as one solution, and the two different Amplifier II primers can be mixed and prepared as the second solution. It is best not to mix the four primers together during the amplification process. This is because, as described in the “Designs of Primers” section, mixing Amplifier I and Amplifier II would allow them to form hybrids in the solution, which reduces the concentration of the primers going to the “target-probe” complex for signal amplification. With the two solutions, signal amplification is carried out by alternately incubating the “target-probe” complex with each solution for approximately one to two minutes, and repeating the “two-step” amplification for as many cycles as possible. This can be done in an automatic manner. After stopping amplification and washing away non-specific binding under stringent conditions, the target is revealed. This can be realized through exposure to X-ray film when radioactive labeling or chemiluminescence detection is used. Alternatively, it can be realized by enzyme-substrate reaction when non-radioactive labeling is used or by fluorescence microscopy reading when fluorescent labeling is used.

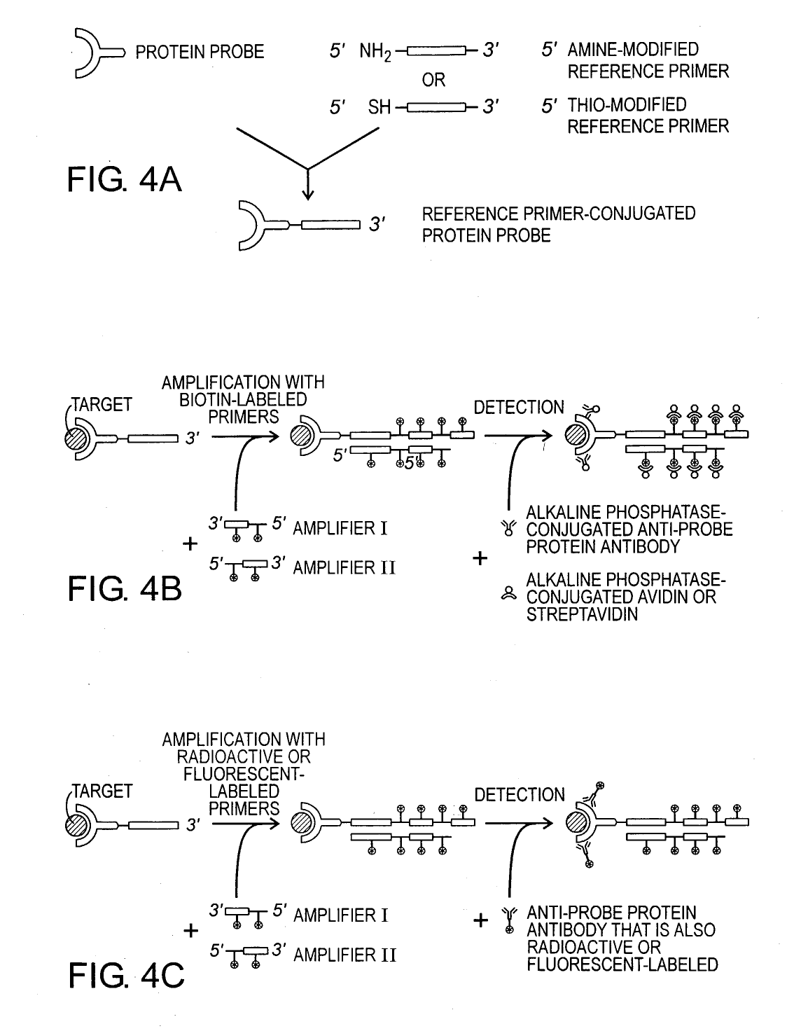

D. Example of Application of the Disclosed Method to Immunoassays (FIG. 4):

A number of bio-assays and molecular detection techniques are based on immunochemical reactions, particularly antibody-antigen interactions. These include protein analysis by Western blotting, antigen detection by ELISA and/or immunohistochemical staining, and the recently developed protein chip technology, etc.

As with nucleic acid based detections, a big challenge for protein-based bio-assays is how to increase their sensitivity to be able to detect the lowest amount of the target molecules. FIG. 4 illustrates how the disclosed method can be used to amplify signals in an immunochemical assay (antigen detection using a specific antibody).

As the first step (FIG. 4A), a reference primer must be covalently linked to the probe, the antigen-specific antibody molecule. This can be realized a number of ways, including chemical cross-linking using either 5′ amine-modified oligo primer or 5′ thiol-modified oligo primer (see description in the “Designs of Primers” section).

Once this DNA-conjugated antibody is available, an immunoassay (FIG. 4B or C) can be conducted by incubating the antibody with the target (antigen). Prior to the incubation, this latter might have been immobilized onto a solid-support such as a membrane (Western blot), or captured by a specific antibody that had been immobilized on the surface of a plastic plate (ELISA) or slide (protein chips). After incubating and washing away non-specific binding, the “antigen-antibody” complex is detected by a secondary antibody that is raised against the first (antigen-specific) antibody in a different animal species. This secondary antibody is either conjugated to a functional enzyme, such as alkaline phosphatase or horseradish peroxidase, or to a fluorescent molecule or other labeled molecule.

In order to carry out signal amplification using the disclosed method, different strategies may be used, depending on how the second antibody is labeled and which detection method is used. If the second antibody is conjugated to an enzyme, such as alkaline phosphatase or horseradish peroxidase (FIG. 4B), a plausible solution to make detection of the DNA amplification signal compatible with that of the antibody labeling system is the use of biotin. In this case, the two amplification primers, Amplifier I and Amplifier II, should be labeled with biotin. Following incubation of the antigen with the reference primer-conjugated antibody from FIG. 4A and the washing away of non-specific binding, signal amplification is conducted as described above. This involves many cycles of alternate incubation of the “antigen-antibody” complex with Amplifier I and Amplifier II primers, with or without a DNA ligase. After amplification and washing away of non-specific binding, the whole complex is then co-incubated with the enzyme-conjugated secondary antibody that binds to the first antibody, and the same enzyme-conjugated avidin or streptavadin that binds to the biotin labels on the DNA hybrids. After washing away non-binding molecules, the target (antigen) is detected by subjecting the entire complex to the enzyme's substrate for color or chemiluminescence reaction.

If the secondary antibody is directly labeled with a radioactive or fluorescent label (FIG. 4C), then the Amplifier I and Amplifier II primers must be labeled the same way. After signal amplification and subsequent washing, the target can be detected on X-ray film or under fluorescence microscope/scanner.

Other application examples of the disclosed method include ligand-receptor binding assays, adhesin-cell binding assays, etc. Regardless of the nature of the assays, it is important to note that it is always the probe molecules that need to be conjugated to the reference primer for signal amplification. Methods to conjugate nucleic acid molecules to the probe molecules depend on the chemical nature of the probes. The same amplification procedure used for nucleic acid hybridization (C) and immunochemical detection (D) can be followed.

E. Simultaneous Signal Amplification of Multiple Probes/Targets:

Many bio-assays, particularly in situ detection and immunohistochemical staining, use multiple probes that are differentially labeled to simultaneously detect different targets. To amplify signals of the different “probe-target” complexes using the disclosed method, a separate set of reference primer and amplification primers is specifically designed for each of the probes. Also it is important that each set of amplification systems and its corresponding probe for signal amplification must be fully compatibly labeled.

For example, in an in situ gene expression analysis, target 1 is cytoplasmic mRNA of gene X, and target 2 is cytoplasmic mRNA of gene Y. A cDNA probe for gene X mRNA is labeled with fluorescent Cy3 and attached at one or both ends with a reference primer for signal amplification. The amplification primers, Amplifier I and Amplifier II, for this probe must also be labeled with Cy3. Similarly, a cDNA probe for gene Y mRNA is labeled with fluorescent Cy5 and attached at one or both ends with a different reference primer. The amplification primers for gene Y probe are also labeled with Cy5. Signal amplification for both probes can be done simultaneously. Preferably, the two differentially labeled Amplifier I primers are mixed as one hybridization buffer, and the two differentially labeled Amplifier II primers are mixed as another hybridization buffer. The sample (target-probe complexes) is then subjected to the two buffers alternately for multiple cycles of amplification, under sufficiently stringent hybridization conditions. After washing, the Cy3 and Cy5 signals can be scanned and detected with different wavelengths.

The same strategy can be used for overall differential gene expression monitoring using DNA chips. Such a test is widely used to monitor overall changes in gene expression of an organism or cells under a different environmental or physiological condition (experimental condition). In general, on the chips, there are thousands and thousands of DNA spots arrayed in a very well defined pattern, with each spot representing an individual gene. To do the analysis, total mRNAs from the sample treated under an experimental condition are converted into single-stranded cDNAs by reverse transcriptase, and are labeled with a fluorescent, such as Cy3, during the cDNA synthesis. At the same time, an equal amount of total mRNAs from a normal sample (control) are also converted into single-stranded cDNAs, which are labeled with another fluorescent, such as Cy5. The two probes are then co-incubated with the DNA chips. Genes that undergo no changes in expression are equally labeled with the two different fluorescents, whereas genes that undergo changes in expression are differentially labeled. As with other bio-assays, very often DNA chips can not detect messenger RNAs of extremely low copy numbers. In this case, signal amplification is very useful. In a preferred scenario, to amplify signals of the two differentially labeled total cDNAs probes on DNA chips, two sets of reference primer and amplification primers are needed. One reference primer is directly attached to a polyT primer for cDNA synthesis of the experimental sample, and a different reference primer is directly attached to a polyT primer for cDNA synthesis of the normal sample. Also, one set of amplification primers is labeled with Cy3 for signal amplification of the cDNA probes from the experimental sample. The other set of primers is labeled with Cy5 for signal amplification of the cDNA probes from the normal sample. Then, Cy3-labeled Amplifier I (set 1) and Cy5-labeled Amplifier I (set 2) primers are mixed to form one signal amplification (hybridization) buffer, and Cy3-labeled Amplifier II (set 1) and Cy5-labeled Amplifier II (set 2) are mixed to form the second hybridization buffer. After co-incubation of the two cDNA probes with the DNA chip, and after subsequent washing, the chip is subjected to the two buffers alternately for multiple cycles of signal amplification, under sufficiently stringent conditions. As described above, fluorescent microscopy or a laser scanner with different wavelengths detects the two fluorescent signals.

In the case of immunohistochemical staining using differently labeled probes, the same strategy is used. For instance, probe I is an antibody that is labeled with Cy3 to detect antigen X, and probe II is another antibody that is labeled with Cy5 to detect antigen Y. In a preferred scenario, each of the two antibodies is attached with a different reference primer, and each has a specific set of signal amplification primers that are compatibly labeled. Simultaneous signal amplification for two probes can be done the same way as described for differential DNA labeling.

Finally, if more than two probes are differentially labeled in a bio-assay, simultaneous signal amplification for all probes can be done in a similar way. What is important, is that for each differentially labeled probe, a separate and compatibly labeled system.

1. A method of amplifying molecular bio-assay signals, comprising the steps of:
introducing a reference primer that can be covalently linked to probe molecules used to detect target molecules in bio-assays which forms a target-probe complex;
introducing two amplification primers, Amplifier I and Amplifier II, the Amplifier I primer being a symmetrical molecule, with its 5′ half sequence fully complementary to the reference primer, and its 3′ half fully complementary to the 3′ half of the Amplifier II primer; the Amplifier II primer also being a symmetrical molecule, with its 5′ half sequence identical to the reference primer and complementary to the 5′ half of Amplifier I, and its 3′ half complementary to the 3′ half of Amplifier I; both Amplifier I and Amplifier II being labeled, the labeling being completely compatible with the probe labeling; and
amplifying the molecular bio-assay signal by incubation of the target-probe complex with Amplifier I and Amplifier II amplification primers.

2. The method as defined in claim 1, the reference primer being a single-stranded molecule.

3. The method as defined in claim 1, the reference primer being one of a oligonucleotide, DNA molecule, RNA molecule, derivatives thereof, or analogs thereof.

4. The method as defined in claim 2, the reference primer having a length of at least 25 bases.

5. The method as defined in claim 4, the reference primer being between 25 and 50 bases in length.

6. The method as defined in claim 1, the reference primer being covalently linked to the probe molecules through one of UV cross-linking, chemical cross-linking, molecular cloning and recombination, or direct attachment during probe synthesis.

7. The method as defined in claim 1, the two amplification primers, Amplifier I and Amplifier II, both being single-stranded molecules.

8. The method as defined in claim 1, the two amplification primers, Amplifier I and Amplifier II, both being one of oligonucleotides, DNA or RNA molecules, derivatives thereof, or analogs thereof.

9. The method as defined in claim 7, the two amplification primers having a length of at least 50 bases.

10. The method as defined in claim 9, the two amplification primers having a length of between 50 and 100 bases.

11. The method as defined in claim 1, both Amplifier I and Amplifier II being labeled with one of a fluorescent label, phosphorescent label, enzymatic label, chemical label, biotin, digoxigenin, or a radioactive label.

12. The method as defined in claim 1, the probe molecules being one of a nucleic acid, protein, peptide, carbohydrate, lipid, hormone, derivatives thereof or analogs thereof.

13. The method as defined in claim 1, the probe molecules being labeled with one of a fluorescent label, phosphorescent label, enzymatic label, chemical label, biotin, digoxigenin, or a radioactive label.

14. The method as defined in claim 1, the target molecule being a chemical.

15. The method as defined in claim 1, the target molecule being one of nucleic acids, proteins, peptides, carbohydrates, lipids, hormones, antibodies or antigens.

16. The method as defined in claim 1, the target molecules being an organism.

17. The method as defined in claim 16, the organism being one of viruses or bacteria.

18. The method as defined in claim 1, the molecular bio-assay signal being amplified by multiple cycles of alternate incubation of the target-probe complex.

19. The method as defined in claim 18, the alternate incubation with the amplification primers being done manually.

20. The method as defined in claim 19, the alternate incubation with the amplification primers being done automatically with a programmable cycle amplification machine (incubator).

21. The method as defined in claim 1, the signal amplification being performed in a single-step incubation, with the two amplification primers mixed together.

22. The method as defined in claim 1, the signal amplification being performed in the absence of any functional enzyme in the signal amplification (hybridization) buffer.

23. The method as defined in claim 1, the signal amplification being performed with functional enzymes in the signal amplification buffer;

24. The method as defined in claim 23, the functional enzymes being enzymes that help to stabilize the amplified signals by making covalent linkage with the probe-target complex.

25. The method as defined in claim 23, the functional enzymes being enzymes that increase the efficiency and speed of the disclosed amplification process.

26. The method as defined in claim 23, the functional enzymes being DNA ligase.

27. The method as defined in claim 1, the amplification primers being used with one of a modified backbone, a modified sugar moiety, or a modified base.

28. The method as defined in claim 1, simultaneous signal amplification being performed on multiple probe-target complexes in a single assay, with a single pair of amplification primers.

29. The method as defined in clam 28, the different probes being linked to a common reference primer; after each of them has bound to its specific target, their signals being amplified with a single pair of Amplifier I and Amplifier II primers that are compatible with the reference primer.

30. The method as defined in claim 1, simultaneous signal amplification being performed on multiple probe-target complexes in a single assay, with different pairs of amplification primers.

31. The method as defined in claim 30, the different probes being for different targets and being differentially labeled and attached with different reference primers.

32. The method as defined in claim 30, in the signal amplification buffer, there being different pairs of Amplifier I and Amplifier II, with each pair of amplification primers being specific to one of the probes.
